(19) United States
(12) Patent Application Publication
(10) Pub. No.: US 2024/0315793 A1
(43) Pub. Date: Sep. 26, 2024

(54) INPUT ARM FOR CONTROL OF A SURGICAL ARM

(71) Applicant: MOMENTIS SURGICAL LTD., Or-Yehuda (IL)

(72) Inventors: Yaron LEVINSON, Or-Yehuda (IL); Idan ROTEM, Or-Yehuda (IL); Hazan BEN, Or-Yehuda (IL); Philip MITSEL, Or-Yehuda (IL); Nelly SOKOL, Or-Yehuda (IL); Shay KATZ, Or-Yehuda (IL); Daniel BOROVOY, Or-Yehuda (IL)

(21) Appl. No.: 18/579,375

(22) PCT Filed: Jul. 14, 2022
(86) PCT No.: PCT/IL2022/050763

§ 371 Date: Jan. 15, 2024

Related U.S. Application Data
(60) Provisional application No. 63/221,988, filed on Jul. 15, 2021.

(51) Int. Cl. A61B 34/00; A61B 34/37

(52) U.S. Cl. CPC A61B 34/74; A61B 34/37

(57) ABSTRACT

A method of operation of an input arm for control of a surgical mechanical arm, the input arm comprising a plurality of rigid segments coupled to each other by joints, at least one of said plurality of rigid segments shaped and sized to be at least partially held by a user's hand, the method comprising: grasping the input arm and manipulating the input arm by moving the rigid segments relative to each other; wherein during grasping and manipulating, electrical power is supplied to at least one brake configured in each of the joints to unlock the brake from a normally locked state; at any selected position of the plurality of rigid segments, entering a pause mode by ceasing the electrical power supply to the at least one brake in each of the joints to thereby lock each of said joints and maintain the plurality of rigid segments in place.

### FIELD AND BACKGROUND OF THE INVENTION

The present invention, in some embodiments thereof, relates to an input arm for control of a surgical mechanical arm, and, more particularly, but not exclusively, to an input arm comprising a plurality of rigid segments which can be paused and held in place at any selected position.

### SUMMARY OF THE INVENTION

According to an aspect of some embodiments there is provided a method of operation of an input arm for control of a surgical mechanical arm, the input arm comprising a plurality of rigid segments coupled to each other by joints, at least one of the plurality of rigid segments shaped and sized to be at least partially held by a user's hand, the method comprising:
grasping the input arm and manipulating the input arm by moving the rigid segments relative to each other; wherein during the grasping and manipulating, electrical power is supplied to at least one brake configured in each of the joints to unlock the brake from a normally locked state;
at any selected position of the plurality of rigid segments, entering a pause mode; by ceasing the electrical power supply to the at least one brake in each of the joints to thereby lock each of the joints and maintain the plurality of rigid segments in place.

In some embodiments, the method further comprises resuming movement of the input arm directly from the selected position in which the input arm was locked.

In some embodiments, resuming comprises re-establishing electrical power supply to the brake of each of the joints to release the brake.

In some embodiments, sequential rigid segments are coupled to each other by one or both of: a flexion joint which is configured to provide for flexion of the coupled rigid segments relative to each other and a rotation joint which is configured for rotation of each rigid segment about a rigid segment long axis, and wherein each of the flexion joint and rotation joint is independently controllable and lockable.

In some embodiments, entering a pause mode comprises actuating a first interface of the input arm, and wherein the resuming comprises actuating a second interface of the input arm, wherein the first interface and the second interface include different interface types.

In some embodiments, the first interface comprises a push button and the second interface comprises a squeezable lever.

According to an aspect of some embodiments there is provided an input arm for control of a surgical mechanical arm, the input arm comprising:
a plurality of rigid segments, at least one of the plurality of rigid segments shaped and sized to be at least partially held by a user's hand;
a plurality of joints, which each joint couples between sequential rigid segments;
wherein each of the joints comprises a brake which changes configuration between a locking configuration in which relative movement of the sequential rigid segments is prevented and an unlocked configuration in which relative movement of the sequential segments is allowed; wherein the change in configurations is controlled via electrical power supply to the electromechanical brake; and
a controller configured to control the electrical power supply to the electromechanical brake to lock or unlock the joint.

In some embodiments, the plurality of joints comprise at least two joints and wherein the controller is configured to control electrical power supply to the at least two joints simultaneously.

In some embodiments, the brake is normally locked and wherein the controller is configured to enable electrical power supply to unlock the brake.

In some embodiments, one of the at least two joints enables flexion of the sequential rigid segments with respect to each other and the other of the at least two joints enables rotation of at least one of the sequential rigid segment relative to the other.

In some embodiments, the input arm comprises at least 5 rigid segments connected to each other by at least 4 joints.

In some embodiments, the brake comprises at least two portions that are magnetically attracted, and wherein when the electrical power supply is enabled, one of the portions is held away from the other portion, allowing for movement of the other portion.

According to an aspect of some embodiments there is provided an input arm for control of a surgical mechanical arm which includes an end-effecter having portions that are movable with respect to each other, the input arm comprising:
a plurality or rigid segments coupled to each other by joints; at least one of the plurality of rigid segments shaped and sized to be at least partially held by a user's hand;
a lever mounted onto the at least one of the rigid segments which is held by the user's hand, the lever protruding outwardly from a surface of the at least one of the rigid segments and configured to slide along a designated track configured on a surface of the rigid segment, wherein sliding of the lever along the track actuates distancing and approximating of the end-effecter portions to each other, depending on the direction of sliding; wherein a degree of distancing of the end-effector portions is set by an axial extent of the lever relative to the track, and wherein the degree of distancing remains constant as long as the lever is not moved on the track.

In some embodiments, the lever comprises a finger attachment shaped and sized for engaging a user's finger, and wherein pulling on the finger attachment slides the lever along the track.

In some embodiments, the finger attachment is loop shaped.

In some embodiments, there is provided a system comprising:
at least one input arm for example as described herein; and
at least one surgical arm controlled by the at least one input arm, the surgical arm including an end-effector that includes portions which are moveable with respect to each other.

In some embodiments, the end-effecter includes one of: a gripper tool, scissors, tweezers.

According to an aspect of some embodiments there is provided an assembly for a joint of an input arm which controls movement of a surgical mechanical arm, the assembly comprising:
a rotatable central elongate shaft;
a brake comprising at least two portions, where a first portion is coupled to the shaft and rotates along with the shaft and a second portion is positioned and configured to stop rotation of the first portion along with shaft when the second portion is approximated towards the first portion such that friction contact is formed; and
a disc shaped encoder configured to detect a rotational orientation of the shaft; wherein the brake and the disc shaped encoder are co-axially stacked on the central elongate shaft, such that the central elongate shaft extends through a central opening formed in the brake and in the disc shaped encoder.

In some embodiments, the assembly comprises a stopper plate configured to limit an angular degree of rotation of the shaft via one or more protrusions or pins.

In some embodiments, the assembly occupies a cylindrical volume of less than 60 cm\hat{ }3.

In some embodiments, a maximal diameter of the assembly is smaller than a diameter of an input arm rigid segment in which the assembly is positioned.

In some embodiments, the assembly comprises a disc shaped printed circuit board co-axially arranged on the elongate shaft.

According to an aspect of some embodiments there is provided a method of determining undesired release of an input arm which controls movement of a surgical mechanical arm, the input arm comprising a plurality of rigid segments coupled to each other by joints, the method comprising:
assessing one or more parameters of at least one joint of the input;
comparing the assessed joint parameters to a set of stored samples, the stored samples being indicative of undesired release patterns of the joint; wherein the comparing comprises scoring the assessed joint parameters based on their correlation to the stored samples;
based on the comparing, determining undesired release and entering a pause mode in which the at least one joint locks to prevent relative movement of the rigid segments.

In some embodiments, the one or more parameters include: a position of the joint, a velocity of movement of the joint, acceleration of the joint.

In some embodiments, the position includes a position of the joint on a 3D coordinate system.

In some embodiments, the method comprises comprising assessing and comparing the one or more parameters for all input arm joints.

In some embodiments, assessing comprises receiving position data from at least one encoder of the at least one joint.

In some embodiments, determining is finalized within a time period shorter than a time delay between control signals generated by the input arm and actual movement of the surgical mechanical arm.

In some embodiments, comparing comprises implementing a time warping algorithm to which the assessed one or more parameters are inputted.

In some embodiments, the input arm comprises at least one inertial measurement unit embedded within the input arm or mounted on it, and wherein the scoring takes into account acceleration measured by the inertial measurement unit.

According to an aspect of some embodiments there is provided a method of detecting undesired release of an input arm which controls movement of a surgical mechanical arm, the input arm comprising at least one inertial measurement unit embedded within the input arm or mounted on it, the method comprising:
measuring, via the inertial measurement unit, one or both of angular acceleration and linear acceleration of at least a portion of the input arm;
based on the measured acceleration, determining undesired release and entering a pause mode in which relative movement of the rigid segments is prevented.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B show a control console (FIG. 1A) comprising a plurality of input arms for controlling movement of a corresponding plurality of surgical arms (FIG. 1B), according to some embodiments;

FIG. 2 shows an input arm for controlling movement of a surgical arm, according to some embodiments;

FIGS. 3A-D show various user interfaces of an input arm, according to some embodiments;

FIG. 4 is a flowchart of a method for pausing and optionally resuming control of a surgical arm by an input arm, while maintaining the input arm at a selected (optionally, last) position, according to some embodiments;

FIGS. 5A-H2 show various components and construction of an input arm joint, according to some embodiments;

FIG. 6 is a block diagram of input arm circuitry, according to some embodiments;

FIGS. 7A-D illustrate another example of a handle segment of an input arm for controlling movement of a surgical arm, according to some embodiments;

FIG. 8 is flowchart of a method for automatic detection of undesired release of the input arm by a user, according to some embodiments; and

FIGS. 9A-D are examples of input arm configurations, according to some embodiments.

### DESCRIPTION OF SPECIFIC EMBODIMENTS OF THE INVENTION

The present invention, in some embodiments thereof, relates to an input arm for control of a surgical mechanical arm, and, more particularly, but not exclusively, to an input arm comprising a plurality of rigid segments which can be paused and held in place at any selected position.

A broad aspect of some embodiments relates to improved usability of one or more input arms which are controlled by a user (e.g. a surgeon, physician) by providing an ability to hold and optionally lock input arm rigid segments at a selected position. In some embodiments, rigid segments are held and optionally maintained at a selected flexion angle relative to each other. In some embodiments, a rigid segment is held and optionally maintained at a selected rotational orientation relative to a sequential segment and/or relative to a joint coupling the sequential segments to each other.

In some embodiments, when the input arm is paused, a corresponding surgical arm which is controlled by said input arm stops moving as well, optionally remaining at a last selected position which corresponds to that of the input arm.

In some embodiments, movement of the input arm segments is resumed directly from the selected position in a continuous manner, for example so that the user does not need to re-position the input arm, move the input arm into a calibration position, align the input arm relative to a surgical arm position or as such.

An aspect of some embodiments relates to a joint locking mechanism in which a joint that couples sequential rigid segments of the input arm is locked via an electromechanical component such as an electromechanical brake. In some embodiments, the input arm comprises multiple joints (e.g. at least 2, 3, 4, 5 joints). In some embodiments, at least two of the multiple joints are locked simultaneously, for example in response to a signal received from a controller. Optionally, all input arm joints are locked simultaneously, for example when entering “pause” mode in which all input arm segments are held in place. In some embodiments, locking of a joint is performed locally at the joint, for example by mechanical and/or electromechanical components physically located at the joint.

In some embodiments, each of a plurality of joints of the input arm is separately controlled, for example, can be locked independently of the other joints. In some embodiments, a controller of the system (e.g. of the control console which includes the input arm(s)) is programmed to control locking and/or unlocking of each of the plurality of input arm joints. Optionally, the controller is configured to simultaneously lock (or release) all joints unitarily.

In some embodiments, a joint brake is changed between a locked configuration and an unlocked configuration in response to a change in electrical power supply (e.g. electrical current conducted to the joint). For example, in some embodiments, when electrical power supply to the joint brake is provided, the brake is unlocked from its normally locked state; and when electrical power is ceased and the joint is disconnected, the brake locks. When electrical power supply is re-established (such as when entering “resume” mode), the brake unlocks. Alternatively, the brake locks when provided with electrical power supply and unlocks when the electrical power supply is ceased.

In some embodiments, an internal joint assembly is structured to be small enough to fit, for example, inside a portion of a rigid segment housing, yet to be sturdy enough to support the weight of the input arm segments, even (and especially) during pause mode, when, for example, some of the input arm segments may be standing upright or otherwise positioned at an angle which counteracts gravitational forces. In some embodiments, a volume occupied by the joint, for example a substantially cylindrical volume, is smaller than 60 cm\hat{ }3, 50 cm\hat{ }3, 40 cm\hat{ }3 or intermediate, larger or smaller volume. In some embodiments, the assembly size and/or volume occupied by the assembly is different for different joints of the input arm, for example, shoulder joints are larger (e.g. in volume and/or specific dimensions) than elbow joints; flexion joints are larger than rotation joints. In some embodiments, the flexion joints are larger than the rotation joints due to inclusion of movement restricting elements, for example, pins and/or protrusions which limit rotation of a central shaft of the joint.

An aspect of some embodiments relates a co-axial assembly of an input arm joint, in which components of the joint are co-axially arranged on an elongate shaft which rotates to provide the relative movement between the rigid segments that are connected at the joint. In some embodiments, components that are circumferentially arranged about the shaft (for example such that the shaft extends within a central opening formed in each) include: a brake; a PCB which controls the joint components; and in some of the joints, optionally only in an assembly of a joint that provides for flexion of the segments relative to each other, a stopper plate which is shaped and configured to limit rotation of the shaft (e.g. via one or more protrusions and/or pins), which in turn limits flexion between the rigid segments coupled at the joint. In some embodiments, an encoder (optionally, a disc shaped encoder) is positioned about the shaft and is configured for detecting a current rotational orientation of the shaft, from which a position of the segments coupled by the joints may be deduced.

In some embodiments, different joints include a similar assembly construction but may differ from each other, for example, by a size of the components, a range of rotation of the shaft (e.g. as set by the stopper plate, in embodiments in which a stopper plate exists and limits rotation).

In some embodiments, flexion of segments relative to each other and rotation of a segment (e.g. about a segment long axis) are actuated separately, such as via two separate joint assemblies. In some embodiments, each of flexion and rotation are locked separately, for example by locking a brake of their designated assembly.

In some embodiments, a rotation joint and a flexion joint are located adjacent each other, for example with a short rigid input arm segment separating between them. In some embodiments, the rotation joint and the flexion joint are similarly constructed, but differ from each other at least in the orientation of the co-axial assembly. In an example, a central shaft of the rotation joint is positioned in a substantially perpendicular alignment with the central shaft of the flexion joint.

An aspect of some embodiments relates to input arm interfaces structured and arranged to reduce non-intended actuation of the input arm and to thereby potentially improve control of the corresponding surgical arm. In some embodiments, input arm interfaces for actuating opposite functions, for example, pause and resume functions, are configured as different types of interfaces, optionally requiring a different interaction with the user. In some embodiments, the interfaces are located at a distance from each other on the input arm handle portion, optionally on different surfaces of the handle. In an example, an interface for entering pause mode is configured as a push button, for example on an upper surface of the handle; while an interface for resuming movement of the input arm, for example from the paused mode, is configured as a lever that is squeezed by the user (e.g. in the direction of the handle axis). A potential advantage of controlling “opposite” functions via interfaces that differ from each other by one or more of: type of interface, a manner of user engagement, a spatial location on the input handle may include reducing a risk of non-intended actuation.

In some embodiments, an input arm interface is configured to ensure that contact with the user's hand had been established: for example, only when the lever is sufficiently pushed or squeezed by the user, resume mode is entered; in another example, a proximity or contact sensor configured on the input arm handle is configured for detecting presence of the user's hand on the input arm.

In some embodiments, an input arm interface is configured according to the surgical arm function and/or structure being controlled by the interface. In some embodiments, an input arm interface is configured to control distancing and/or approximation of portions of an end-effector of the surgical arm, for example a gripper that includes jaws. In an example, for controlling the gripper, an input arm interface which controls a degree of opening of the jaws includes slidable lever, where movement of the lever along a designated track sets the degree of opening of the jaws. In some embodiments the track is an arched track or is otherwise non-linear. In some embodiments, the track is linear. In some embodiments, the track is defined as a slot in which at least a portion of the lever is seated. Optionally, sliding of the lever in one direction opens the jaws, and sliding of the lever in the opposite direction closes the jaws. In some embodiments, if a user releases the lever at a certain axial position along the track, an opening degree of the jaws remains as set by the lever. This may be advantageous, for example, when grasping tissues using the jaws, so that tissue held in between the jaws may remain grasped and not unintendedly released.

In some embodiments, the lever is moveable between discrete positions defined along the track. Alternatively, the lever is continuously moveable and can be stopped (e.g. when let go of by the user) at any position along the track.

An aspect of some embodiments relates to determining a state of undesired release of the input arm, for example, if the user unintendedly lets go of the input arm handle. In some embodiments, if a determination of undesired release is reached, the system (e.g. the system controller) automatically enters a pause mode, optionally locking the joints of the input arm to hold the input arm segments in place and potentially prevent further fall.

In some embodiments, a determination of undesired release is reached within a time period that is shorter than an inherent delay between a control signal issued by the input arm, and actual movement of the corresponding surgical arm that is controlled by the input arm. A potential advantage of determining and optionally responding to (such as by entering pause mode) a situation of undesired release within a time period shorter than a delay between the input arm control signal and actual movement of the surgical arm may include that the surgical arm is least or not at all affected by the drop of the input arm, potentially preventing damage to tissue which could have been caused if the surgical arm was to move according to the falling input arm.

In some embodiments, a determination of undesired release is reached with the aid of software algorithms, for example, by comparing detected input arm joints positions and/or segments positions and comparing the detected positions to known (optionally, stored) positions that are indicative of undesired release. In some embodiments, temporal sequences of input arm joint positions (such as on a 3D coordinate system) are compared to samples of measured and/or simulated positions, and if the currently detected sequences sufficiently match the sample sequences, a situation of undesired release is determined. In some embodiments, the comparison is made based on one or more of: a position of the joint, a velocity of movement of the joint, an acceleration of the joint, and/or other movement and/or position related parameters. In some embodiments, one or more parameters are calculated based on other, e.g. joint velocity is calculated as a derivative of a vector of joint positions over time.

In some embodiments, in addition or alternatively to determining undesired release with the aid of software algorithms, an accelerometer or an inertial measurement unit embedded and/or mounted on the input arm are used for measuring linear and/or angular acceleration of input arm portions. If the measured acceleration is higher than a threshold, a situation of undesired release is determined.

In some embodiments, a cumulative determination is made, for example taking into account input arm positions (for example as detected by encoders of the input arm) and acceleration results.

As referred to herein, the term “proximal” may refer to portions or elements (e.g. input arm portions) that are closer to the user end (e.g. to a physician, surgeon, technician and/or other clinical person that is holding the input arm) and/or closer to a portion of the input arm that is shaped and sized for handling by a user. Accordingly, the term “distal” may refer to portions or elements (e.g. input arm portions) that are closer to a base, platform or console from which the input arm extends.

As referred to herein, a position of an input arm segment and/or a position of a joint may include, for example, a spatial position on a 3D coordinate system; a position (such as an angle, a direction of extension) relative to a base (e.g. to the control console platform) and/or relative to the floor and/or relative to the surgical bed; a position relative to a calibrated (optionally, rest) position of the input arm; a position in which the input arm is aligned with a surgical arm position, optionally, with a surgical arm calibration (or rest) position.

In some embodiments, a position of a joint is determined with the aid of an encoder, for example configured at the joint. In some embodiments, a rotational extent of a central shaft of the joint is detected by the encoder (such as relative to a calibration or reference position).

In some embodiments, a position of a rigid segment refers to one or more of: a location of center of the segment; an axially centered location; an angle of the segment relative to a sequential segment; a proximal or distal end if the segment; and/or other.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### A Surgical System Comprising Input Arm(s) for Control of Surgical Arm(s)

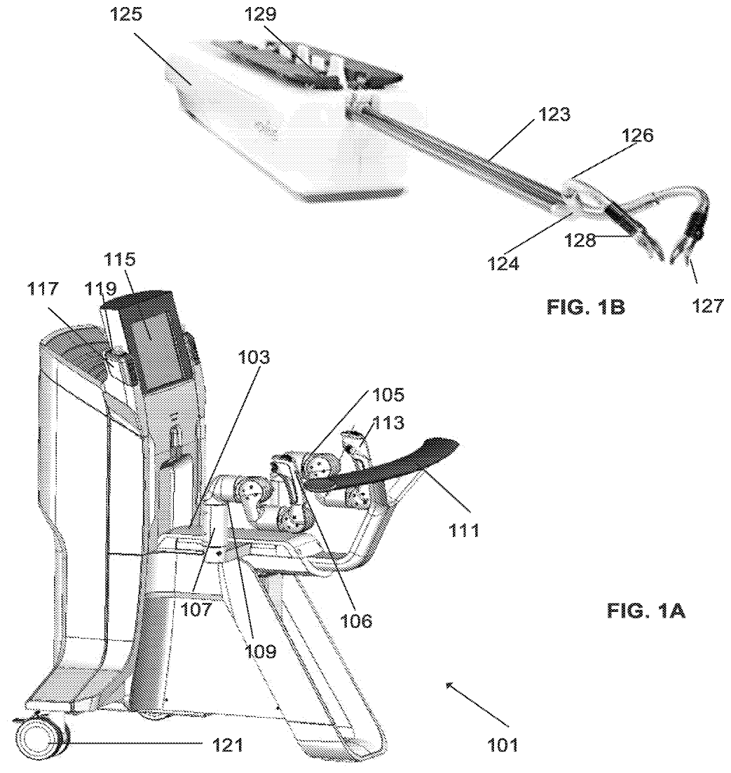

FIGS. 1A-B show a control console (FIG. 1A) comprising a plurality of input arms for controlling movement of a corresponding plurality of surgical arms (FIG. 1B), according to some embodiments.

In some embodiments, control console 101 comprises a platform 103 from which a plurality of input arms such as input arms 105, 106 extend (e.g. 1, 2, 3, 4, 5, or a higher or lower number of input arms). In some embodiments, the input arm is connected to the platform by a support segment 107, which is coupled on one end to the platform and on its other end to a most distal input arm segment 109.

In some embodiments, a position of the support segment with respect to the platform is adjustable, for example during set up of the input arm and/or during use of the input arm. In some embodiments, an angle of the support segment is initialized e.g. to be parallel or perpendicular to the floor.

In some embodiments, a separation between input arms 105, 106 is adjustable, for example by lateral movement of at least one of the input arms with respect to the other and/or with respect to the platform.

In some embodiments, the control console includes a user support, for example in the form of an arm support 111 and/or a seat (not shown). In some embodiments, the user support position relative to the input arm position and/or relative to the platform is adjustable (e.g. by slidable movement relative to the platform which sets a distance between the user support and the input arm (for example, input arm handle 113); by adjustment of a height of the user support relative to the platform).

In some embodiments, during use, a user (e.g. physician, surgeon, nurse or other clinical personnel) rests their arms (e.g. their elbows) on the user support, while holding the input arm handles in their hands. In some embodiments, the control console includes a display 115, for example, a screen display, for providing data to the user and/or for receiving input data from the user. In some embodiments, the screen displays data related to a surgical arm position, data related to an anatomical location of the surgical arm, operation related data, data pertaining to an input arm status or position (e.g. indicating a current mode of activation of the input arm, such as paused/active), patient related data and/or other.

In some embodiments, the control console includes additional user input, for example configured as handles 117 including, for example, thumb contacts 119. In some embodiments, handles are positioned adjacent the screen, e.g. on opposing sides of the screen. In some embodiments, handles 117 provide specific surgical arm control, for example, control linear movement of the surgical arm, optionally during initial stages of insertion into the body.

In some embodiments, the control console is mobile, for example comprising wheels 121 which enable movement of the console on the floor. In some embodiments the control console can be selectively positioned relative of the patient bed and/or relative to the surgical arms and/or motor units which drive movement of the surgical arms. In some embodiments, the control console is positioned relative to an insertion location into the patient body.

FIG. 1B shows a pair of surgical arms 123 extending from a motor unit 125 which houses one or more motors for driving movement of the surgical arms.

In some embodiments, the surgical arm comprises a plurality of segments, where at least some of the segments are flexible and function as joints (allowing flexion and/or extension). In some embodiments, different segments are rotatable about a segment long axis. In an exemplary embodiment, a surgical arm includes a shoulder joint 124, an elbow joint 126, and optionally a wrist joint 128. In some embodiments, distally to the wrist joint, an end effecter 127 (e.g. a gripper tool) is attached.

In some embodiments, a flexible segment of the surgical arm is bendable by at least 120°, or by at least 90, or by at least 100°, or by at least 140°, or by at least 160°, or by at least 180°, or by at least 190°, or by at least 200°, or by at least 2100 or lower or higher or intermediate angles. Optionally, at least one flexible segment allows retroflecting of the surgical arm.

In some embodiments, the surgical arm comprises one or more gears (not shown) which actuate bending and/or rotation of the arm segments. In some embodiments, when the surgical arm is received in the motor unit (e.g. inserted into a designated recess 129 of the motor unit), the surgical arm gears are engaged by the motor(s) of the motor unit.

In some embodiments, the surgical arm is inserted into a patient body. In some embodiments, insertion is via a natural orifice (e.g. vagina, rectum, mouth and/or nostril). In some embodiments, insertion is via an incision. In some embodiments, the surgical arm (commonly, two surgical arms) are used during a gynecological procedure, optionally through transvaginal access. In a specific example, the procedure involves hysterectomy.

Additional examples of surgical arms and control consoles are for example as described in U.S. Pat. No. 10,070,930 which is incorporated by reference herewith.

### General Input Arm Structure

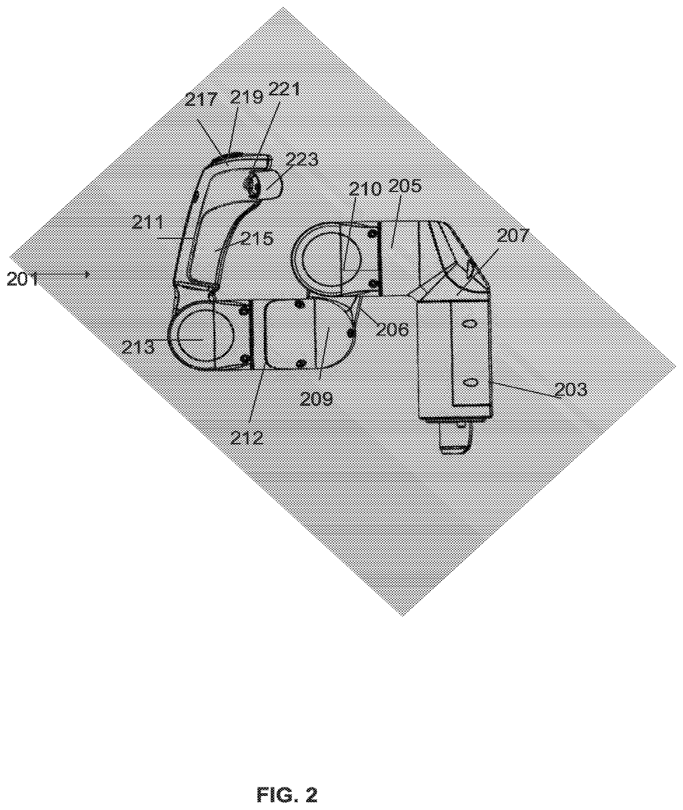

Referring now to the drawings, FIG. 2 shows an input arm for controlling movement of a surgical arm, according to some embodiments.

In some embodiments, an input arm is maneuvered by a user (e.g. a physician, surgeon) to control movement of a surgical arm which is at least partially inserted into the patient body. In some embodiments, an input arm structure corresponds to that of a surgical arm, for example so that movement of input arm segment(s) results in movement of corresponding surgical arm segment(s). In some embodiments, the surgical arm defines a humanoid structure, where segments and connecting portions between the segments are equivalent to those of a human arm, including, for example: a humerus section, a radius section, a shoulder joint, an elbow joint, and a wrist joint.

In some embodiments, in use, movement and/or a position of input arm components (e.g. rigid segments, joints) are mapped to movement and/or a position of corresponding surgical arm components (e.g. rigid portions, flexible portions acting as joints of the surgical arm).

In some embodiments, each driven portion of the surgical arm has a corresponding portion in the input arm. In some embodiments, an input arm includes at least the number of joints and/or segments as a corresponding articulated surgical arm. In some embodiments, the input arm and the surgical arm include the same number of segments and/or the same number of connecting portions.

In some embodiments, one or more portions of the input arm have the same degrees of freedom as that of a corresponding portion of a surgical arm. For example, in some embodiments, input arm segments are bendable by about the same amount as corresponding surgical arm portions. For example, surgical arm portions which are rotatable around the surgical arm portion long axis correspond to input arm segments which are rotatable around the input arm portion long axis. For example, in some embodiments, a flexible surgical arm portion is bendable by the same angle as an angle between two input device segments coupled by a joint corresponding to the flexible surgical arm portion.

In some embodiments, an angle between long axes of input arm segments coupled by a joint controls an angle of a corresponding surgical arm flexible portion, where, for example, an angle of the surgical arm flexible portion is defined between long axis tangents of the flexible portion at the flexible portion ends.

In an exemplary embodiment, an input arm includes a more angular shape and/or a shape with a larger relative lateral extent than that of the surgical arm. For example, in an exemplary embodiment, input arm connecting portions may include pivot connections between rigid segments, whereas surgical arm connecting portions are long bendable (flexible) sections.

In some embodiments, an input arm 201 comprises a plurality of rigid segments (e.g. 2, 3, 4, 5, 6, 8, 10 or intermediate, larger or smaller number of segments), where adjacent segments are coupled to each other at a connecting portion, such as a joint. Optionally, a segment is defined as a rigid portion extending between two sequential joints.

In some embodiments, the segment functions to separate between two sequential joints, for example having a length only long enough so as not to interfere with movement enabled by the joint. In some embodiments, a length of the segment is selected according to the mechanical and/or electrical components housed within that segment.

In an exemplary construction, an input arm includes the following segments and joints: a first (most distal) segment 203 which extends from a platform on which the input arm is mounted (platform not shown); a second segment 205 coupled to the first segment 203 at a first joint 207; a third segment 206 coupled to the second segment 205 at a second joint 210; a fourth segment 212 coupled to the third segment 206 at a third joint 209; a fifth (handle) segment 211 coupled to the fourth segment at a joint 213. In some embodiments, joint 207 functions as a shoulder rotation joint; joint 206 functions as a shoulder flexion joint; joint 209 functions as an elbow rotation joint; and joint 213 functions as an elbow flexion joint.

In some embodiments, the handle segment 211 includes a portion that is shaped and sized for gripping by a user's hand and/or by parts of the user hand (e.g. by at least one finger). In an example, segment 211 includes a surface 215 which ends, at a proximal end thereof, with a bulge 217. Optionally, surface 215 is concave. Optionally, in use, a user's palm is partially wrapped about the concave surface, while the thumb and optionally one or more other fingers such as the index finger are placed on or around the bulge.

In some embodiments, the input arm includes one or more user interfaces, for example formed as push button/s, slide button/s, scroll wheel/s, touch sensitive buttons and/or LCD displays. In some embodiments, an interface operates a certain surgical arm function and/or movement, for example, actuates linear movement of the surgical arm (direct back and forth movement of the surgical arm, optionally carried out by direct back and forth movement of the motor unit to which the surgical arm is attached); actuates rotation of surgical arm portions; actuates movement of a tool configured at a distal end of the arm (e.g. a gripper); allows selection of an operational mode of the input arm and/or of the surgical arm (e.g. pause, resume) and/or other functions.

In some embodiments, a plurality of buttons 219 are mounted on the gripping portion, e.g. on bulge 217. In some embodiments, a lever 221 extends laterally to bulge 217, comprising a loop 223 and/or other attachment shaped and configured for being engaged by a finger (e.g. an index finger) of the user. In some embodiments, the loop facilitates pulling and/or pushing of the lever on its track.

In an example, in use, a user grips the input arm at segment 211, placing their palm at the concave surface 215 and wrapping the finger about the bulge, with the index finger inserted into the loop 223.

In some embodiments, by grasping at least a portion of segment 211, a user manipulates movement of more than one segment of the input arm. For example, the user moves the input arm by pulling segment 211 proximally towards them, by pushing segment 211 distally away from them, by moving segment 211 sideways, and/or by directing segment 211 at any desired angle, for example relative to the base (or console) from which the input arm extends. In some embodiments, movement of segment 211 results in movement of the one or more additional input arm segments, causing the segments to change their relative position with respect to each other at a joint coupling between them.

In some embodiments, a joint coupling between sequential segments is configured to provide for bending (flexion) of at least one of the segments relative to the other; or for rotation of at least one of the segments relative to each other. In some embodiments, the joint is a pivot joint, where depending an orientation of the joint components (e.g. an inner shaft of the joint, as further described herein) enables either rotation of a segment or flexion of a segment relative to its sequential segment.

In some embodiments, as further detailed herein, a joint is configured for locking. In some embodiments, locking prevents movement of the coupled segments relative to each other. In some embodiments, locking maintains the coupled segments at the last position of the segments (e.g. at the segment position measured and/or detected right before locking was applied, or the segment position measured and/or detected during the applying of locking). In some embodiments, a joint is locked selectively, optionally independently of other joints. Alternatively, all joints of the input arm are simultaneously locked (such as when entering “pause mode”, as further detailed herein).

In some embodiments, an input arm structure has one or more ratios and/or dimensions which match a corresponding ratio and/or dimension (optionally scaled) of a surgical arm and, optionally, one or more other dimensions and/or ratios which do not match those of a surgical arm. In an example, a length ratio between two segment lengths of an input arm and a surgical arm are substantially the same, for example, with 0-5%, or 0-1%, or 0-0.5%, or lower or higher or intermediate ranges or values of a difference between the ratios.

In some embodiments, one or more matching segment length ratios between an input arm and a surgical arm enable intuitive control of the surgical device with the input arm, for example, despite structural differences between the arms. In some embodiments, segment length ratios between the input arm and surgical arm match, but actual segment length ratios do not match. For example, in some embodiments, a surgical arm includes long connecting portions (e.g. flexible portions), and an input arm capable of controlling the surgical arm includes short connecting portions for example, local joints. Potentially, an advantage being ease of control of the input arm (e.g. input arm segments move freely about joints) while a surgical arm maintains an elongate, “corner-less” structure without pointy angles.

In some embodiments, a thickness of one or more input arm segments (e.g. a diameter of cylindrical segments and/or largest segment cross sectional dimension) is different (e.g. larger) than to that of a surgical arm. Increased input arm segment thickness potentially provides space for sensors and/or locking devices and/or provides an input arm with dimensions which are comfortable and/or easy for a user to maneuver or grasp.

Exemplary input arm dimensions may include: an axial length of between 200-300 mm, 150-250 mm, 230-280 mm or intermediate, larger or smaller range (measured for example between the proximal end and the distal end of the input arm, when all joints are fully straightened, such that a 0 degree angle exists between sequential input arm segments); a maximal width (measured for example at a widest rigid segment cross section, e.g. diameter) of between 50-100 mm, 20-200 mm, 30-80 mm or intermediate, larger or smaller width.

### Exemplary User Interfaces of an Input Arm

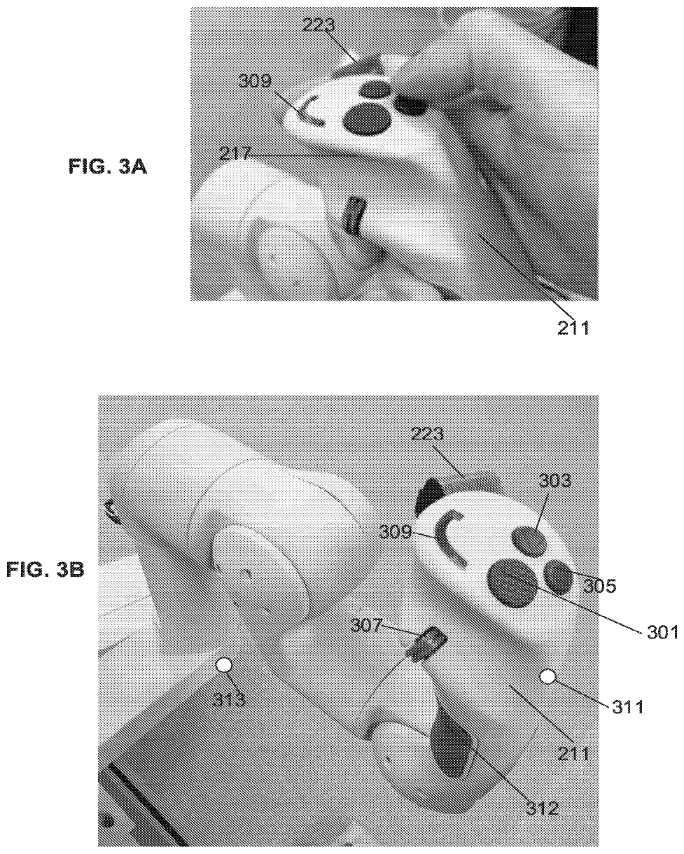

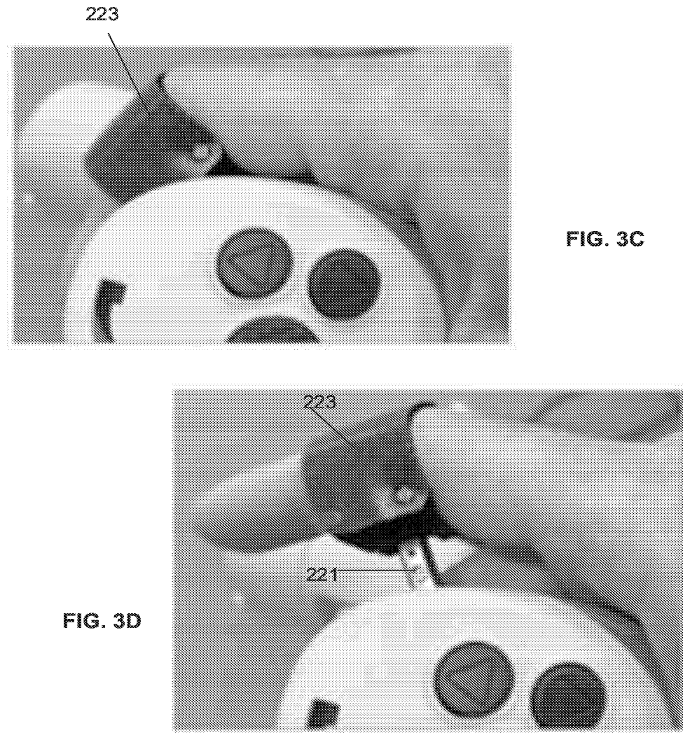

FIGS. 3A-D show various user interfaces of an input arm, according to some embodiments.

In some embodiments, at least a portion of the input arm, such as at least a portion of proximal handle segment 211 is shaped and sized for engagement by a user hand. In some embodiments, as shown in the example of FIG. 3A, a user holds the input arm for example by placing their thumb on top of bulge 217, their middle finger abutting against a bottom surface of the bulge, and optionally, their index finger inserted into loop 223 of lever 221 (shown best in FIG. 3D). In some embodiments, at least a portion of segment 211 is covered by an anti-slip surface 312 (e.g. a rugged surface and/or a surface formed of a material which improves friction with the user's hand, e.g. rubber).

In some embodiments, as shown for example in FIG. 3B, an input arm proximal segment 211 comprises one or more of the following interfaces:
A plurality of tactile push buttons, for example button 301 for entering pause mode; button 303 for advancing the surgical arm linearly forwards, button 305 for retracting the surgical arm linearly backwards. Optionally, the buttons are mounted on a top surface of bulge 217 to be easily accessible, for example pressed on by the user's thumb, and easily visible. Optionally, the buttons remain easily accessible at all input arm angles and positions, facing towards the user. In some embodiments, the buttons are formed of or comprise a material which improves friction contact with the user's hand, for example, the button surface is formed of rubber. In some embodiments, a button is seated directly onto its control circuitry (such as a PCB), for example without interfacing elements such as a spring). In some embodiments, a pushed button remains pressed down without requiring the user to maintain hold of the button. In some embodiments, button actuation (e.g. for entering “pause mode”) is by a short single push of the button.
A scroll knob 307 which actuates, for example, rotation of a wrist joint of the surgical arm (a joint to which an end tool such as grippers is attached, for example). Optionally, the scroll knob is located at a bottom surface of bulge 217, and can be engaged, for example, by the middle finger or thumb of the user;
A LED indicator 309, which for example changes color depending on the current operational mode of the input arm (such as: blue light for pause mode, green light for active/resume mode, red for indicating an error or misalignment);
A lever 221 including the loop 223, which is used, for example, for operating an end tool of the surgical arm, such as for opening a gripper when slid in one direction (for example as shown in FIG. 3C) and for closing the gripper when slid in the opposite direction (for example as shown in FIG. 3C) with respect to the bulge 217.
   In some embodiments, a relative position of the lever with respect to a track or slot along which the lever is moved is indicative of a current state of the end tool (e.g. a gripper, scissors, tweezers, a needle holder and as such). In some embodiments, the end tool is maintained at a selected state (e.g. a selected degree of opening of gripper jaws) by holding lever 221 in place relative to the track, either by the user keeping their finger inside the loop and/or by simply letting go of the loop, allowing it to remain at its current position on the track. In some embodiments, the end tool configuration remains unchanged unless the lever is pulled or pushed by the user.
   In some embodiments, lever 221 is resilient so that loop 123 can be pushed (e.g. squeezed) by the user towards the bulge 217. In some embodiments, this squeezing movement actuates entry into resume mode (for example, from a paused mode). Optionally, squeezing ends with a “click” sensational feedback to the user.
   A potential advantage of entering resume mode in response to applying of force by a user, such as by squeezing of the loop, may include that resume mode is entered only when intended contact is made between the user the input arm, reducing or preventing unintended actuation of the input arm. In addition, the different types of interfaces for entering pause mode and for entering resume mode and their physical separation (their locations on the input arm) may be potentially advantageous in that unintended actuation or confusion between the two modes may be reduced or prevented.

Going back to FIG. 3B, in some embodiments, the input arm includes one or more sensors 311 for detecting that a hand of the user is in proximity and optionally in contact with the input arm. In some embodiments, as a safety consideration, the input arm may be locked for use if presence of user's hand is not detected. In some embodiments, the sensor(s) are located on proximal segment 211, for example on a proximal surface of bulge 217, such as where a palm of the user's hand rests against. Examples of hand presence sensors may include light sensors, IR sensors, force sensors (e.g. pressures sensors) and/or any other sensor suitable for detecting that a user's hand is sufficiently close to the input arm to enable operation of the arm (e.g. less than 5 cm, less than 2 cm, less than 1 cm close) and/or that the user's hand is in contact with the input arm, for example at least partially resting against the input arm.

In some embodiments, the input arm comprises one or more vibrating elements, e.g. vibration motors (not shown), for example embedded inside the segments of the input arm and/or mounted onto the segments. In some embodiments, the vibrating elements are configured to vibrate one or more portions of the input arm to provide feedback to a user holding the input arm. For example, vibration indications (which optionally differ in strength and/or duration and/or location on the input arm) are provided in response to specific input arm actuations (e.g. in response to pressing of buttons for actuation of linear movement, when lever 221 is slid), when an operational error occurs, when the input arm is misaligned (for example does not match a current surgical arm configuration), and/or other situations in which feedback in the form of vibration may assist the user in determining an input arm status or position.

In some embodiments, the input arm and/or the console from which the input arm extends includes a technician button 313 which, when activated, is configured to release locks of the input arm, such as when the system is turned off, enabling to move the input arm (without resulting in movement of the surgical arm). In some embodiments, the technician button is used for maintenance purposes (e.g. input arm repair) and/or if the system malfunctions.

The table below lists some examples of surgical arm actions and the respective interface and user action carried out on by manipulation of the input arm:

| Action | Control | Action of user |
|---|---|---|
| Unlock JS, resume Arms | lever | Squeeze |
| | | And grips are matched |
| Close/open jaws | lever | Close and open |
| Lock JS, pause Arms | Pause button | Press |
| Wrist rotation | Roller | Scrolling the roller's knob |
| Forward Linear | Forward button | press |
| movement | | |
| Backward Linear | Backward button | press |
| movement | | |

In some embodiments, the input arm is ergonomically structured, for example by the interfaces being placed at input arm positions which prevent or reduce discomfort to the user. For example, by having the buttons mounted on the top surface of the bulge, discomfort to the wrist of the user may be reduced or prevented, especially when bending the input arm in various angles that are different from a straight up vertical orientation.

In some embodiments, the input arm is constructed such that a weight of the input arm segments is supported by the joints, for example, by the one or more brakes of a joint.

### Exemplary Pausing and Resuming Movement of an Input Arm

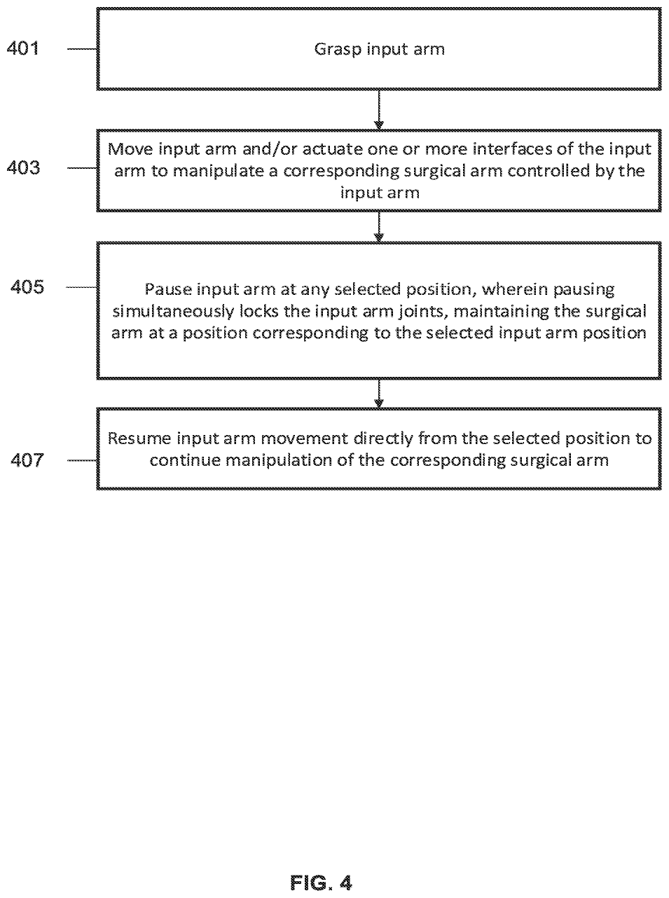

FIG. 4 is a flowchart of a method for pausing and optionally resuming control of a surgical arm by an input arm, while maintaining the input arm at a selected (optionally, last) position, according to some embodiments.

In some embodiments, a user grasps an input arm (401), for example by placing their hand in contact with at least a segment (e.g. with a most proximal segment) of the input arm.

In some embodiments, the user moves the input arm and/or actuates one or more interfaces of the input arm to manipulate a corresponding surgical arm which is controlled by the input arm (403).

In some embodiments, input arm movement includes bending and/or rotation of input arm segments relative to each other and/or relative to the console. Movement of input arm segments and/or joints is detected by the system, for example via encoders embedded at the joints, and the movement is optionally translated to similar movement of the surgical arm portions corresponding to the input arm portions.

In some embodiments, the user and/or the system decide to pause the input arm at a certain position to thereby hold the surgical arm at the same selected position (405).

An automated (system controlled) decision to pause the input arm may be obtained, for example, if drop of the arm is detected and/or if presence of a user's hand is not detected (for example by the proximity sensor).

A user initiated decision to pause the input arm may be obtained, for example, in situations in which the user wants to relax their hand, decide on a next move, allow for a certain surgical arm function or change (e.g. a change in a distal end tool of the surgical arm), and/or other situations.

In some embodiments, when two input arms are manipulated by the user, if a user decides to use only one input arm (e.g. for a certain time period), the other input arm may be paused during that time period.

In some embodiments, the user enters pause mode by actuating an interface of the input arm, for example, by pushing a button. In some embodiments, in pause mode, at least one and optionally all of the input arm joints lock, preventing any bending and/or rotation at the joint. In some embodiments, all input arm joints are locked simultaneously.

In some embodiments, as further detailed below, an input arm joint comprises a brake, and when entering pause mode, electrical supply to the brake is ceased, causing portions of the brake to cling to each other and thereby restrict or prevent movement of the joint. Optionally, disconnecting of electrical supply to the joint brakes is timed so that all joints of the input arm are locked altogether, and the input arm is maintained at the selected position, optionally being the last position set by the user. In some embodiments, the brake is an electromagnetic brake, which is optionally normally closed (locked to prevent movement).

In some embodiments, during pause mode of the input arm, the surgical arm is paused in a corresponding position. In some embodiments, during pause mode of the input arm, the surgical arm is disconnected for example so that no control signals are sent from the input arm and/or are received at the surgical arm.

In some embodiments, input arm movement is resumed, allowing to continue manipulation of the corresponding surgical arm (407). In some embodiments, resume mode is entered by actuation of an input arm interface, for example by squeezing of a lever, as described herein. In some embodiments, in resume mode, electrical supply to the joints brakes is re-established, releasing the brakes and unlocking the joints.

Some potential advantages of a pause mode in which input arm joints can be locked at any selected position, holding input arm segments at a certain angle between them and/or at a certain spatial location (for example as measured on a 3 dimensional coordinate system) may include that the user may stop and optionally release hold of the input arms at any time, without being required to place the arms at a predefined position (e.g. a calibration position). When regaining control, input arm movement can be resumed continuously from the position in which the input arm was paused.

In some embodiments, when two (or more) input arms are used, an input arm can be independently and selectively paused, while the other input arm(s) remain active.

### Exemplary Input Arm Joint Structure

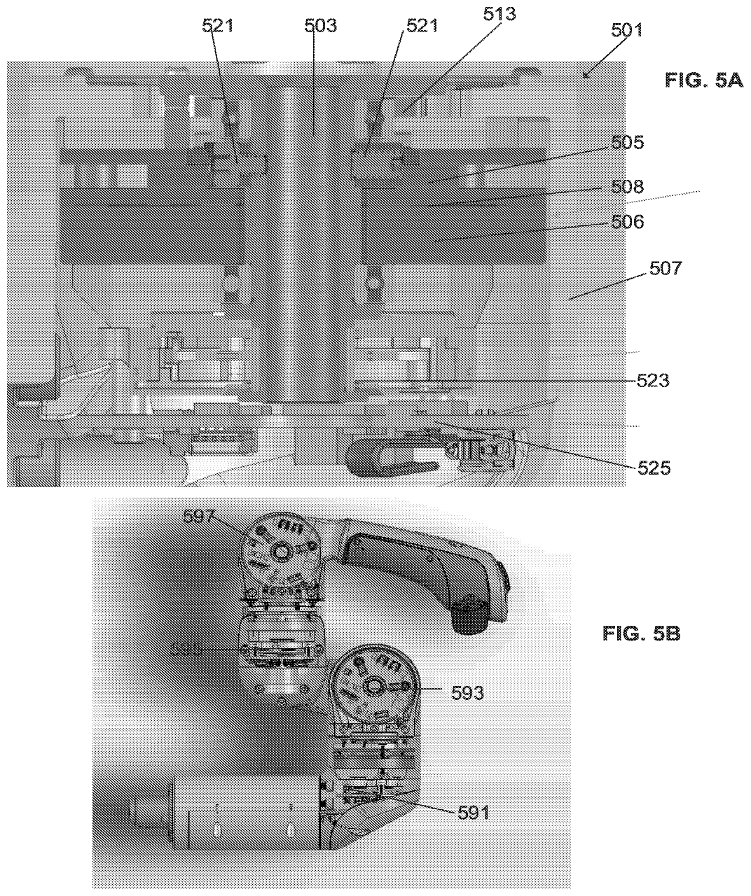

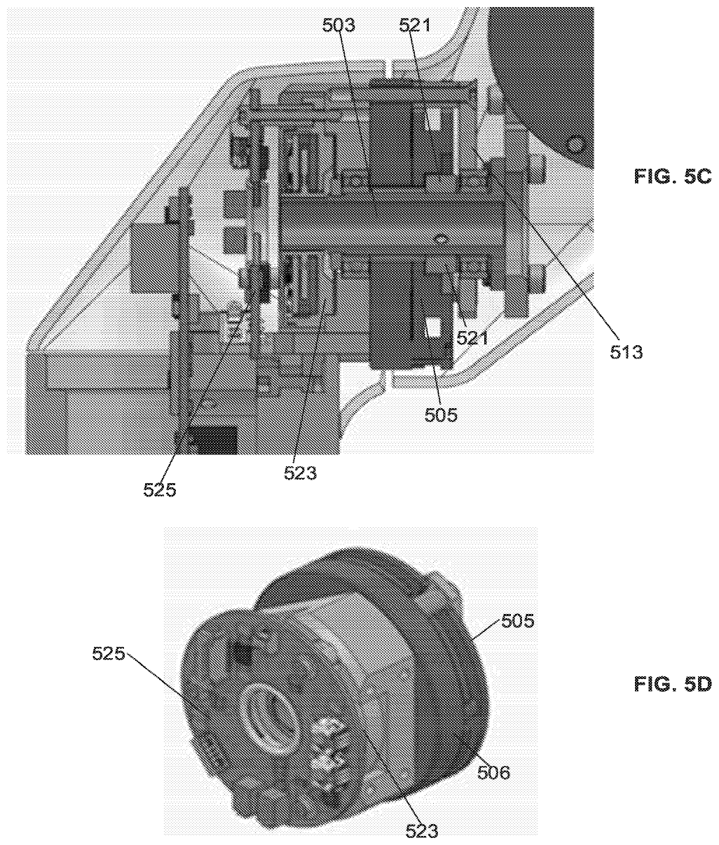

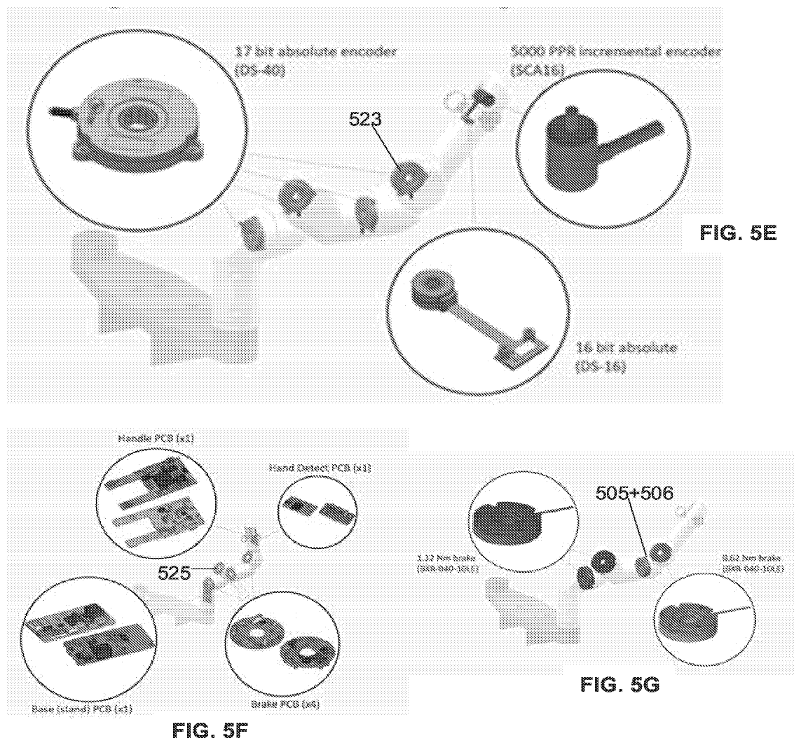

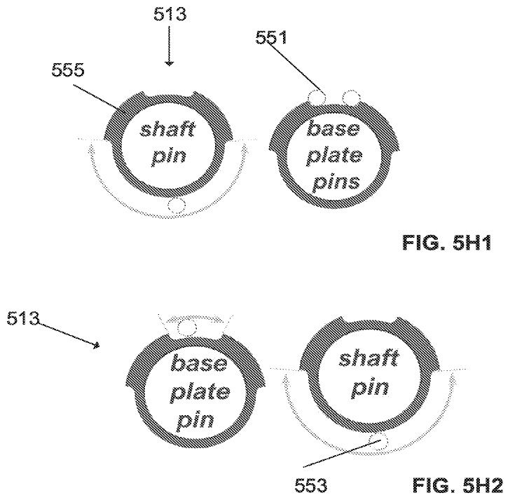

FIGS. 5A-G show various components and construction of an input arm joint, according to some embodiments.

In some embodiments, an input arm joint 501 comprises movement control components and/or computational components housed within external walls 507 (such as a housing or a shell) of the input arm segments, at a coupling between the segments.

In some embodiments, joint 501 comprises a central shaft 503 (for example, a cylindrical shaft). In some embodiments, shaft 503 is configured to rotate about the shaft long axis.

In some embodiments, shaft 503 extends axially through a central opening of a brake, for example a brake formed of two or more portions 505, 506. In some embodiments, the brake comprises an electromagnetic brake, which is activated using electrical power supply, and configured to stop rotational movement of shaft 503.

In some embodiments, shaft 503 is coupled to brake portion 505 by a brake coupling 521, such that brake portion 505 rotates along with the shaft 503 when the shaft rotates about its long axis.

In some embodiments, when no electrical power is supplied to the brake, portions 506 and 505 cling to each other, such that portion 506 prevents portion 505 from rotating along with the shaft 503. In some embodiments, clinging movement of portion 506 of the brake to portion 505 of the brake is spring actuated (spring not shown). In some embodiments, a magnetic attraction exists between portions 505 and 506.

In some embodiments, when electrical power is supplied to the brake, portions 505 and 506 are held apart from each other, for example being spaced by a spacing 508 which enables rotation of brake portion 505 along with shaft 503.

Examples of brake positions in each of the input arm joints are shown in FIG. 5G.

In some embodiments, a stopper plate 513 is positioned about the shaft and configured to limit the extent of rotation of shaft 503. In some embodiments, rotation is limited by one or more pins or protrusions of the shaft and/or of the stopper plate. In some embodiments, as shown for example in FIG. 5H1-2, a rotational extent of the shaft is limited a pin arrangement for example as follows: in FIG. 5H1, demonstrating for example an extent of rotation of a shoulder flexion joint, a set of pins (a single pin indicated by 551) limits rotation of the shaft 503 to an extent of, for example, 180 degrees; in FIG. 5H2, demonstrating for example an extent of rotation of an elbow flexion joint, a set of pins (a single pin indicated by 553) limit rotation of the shaft to an extent of, for example, 215 degrees. In some embodiments, a restricted rotational extent is obtained by having the pin(s) encounter resistance by either encountering another pin and/or by a protrusion (such as a radial protrusion 555) for example formed in the stopper plate 513.

In some embodiments, joint 501 comprises an encoder 523 configured for measuring one or more of a rotational orientation of the shaft, a speed of rotation of the shaft, a count of shaft rotations, and/or other parameters, to identify a current state of the joint for determining an absolute and/or relative position of the input arm segments which are coupled by the joint. Optionally, the encoder measures the rotational position, and one or more other parameters are calculated therefrom (e.g. by a system controller).

In some embodiments, encoder 523 is disc shaped and configured radially externally to the shaft 503. Examples of encoders positions in the input arm joints are shown in FIG. 5E.

In some embodiments, encoder 523 is selected to reduce or avoid electromagnetic interference.

In some embodiments, components of the joint are arranged co-axially about the shaft 503. Optionally, shaft 503 extends through central openings formed in the brake portions 505 and 506, the encoder 523 and/or the stopper plate 513. A potential advantage of a co-axial assembly may include that the joint components occupy a relatively small volume, which is fitted within a substantially cylindrical housing defined by the walls of the rigid input arm segments.

In some embodiments, joint 501 comprises circuitry components, such as a printed circuitry board (PCB) 525. Examples of PCB positions in the input arm joints and segments are shown in FIG. 5F. In some embodiments, the PCB is maintained (e.g. by fixation elements, such as screws or pins, and/or by adhesives (e.g. glue)) in place and does not rotate along with the shaft 503 and brake portion 505.

In some embodiments, different input arm joints have the same general construction but may differ from each other in one or more of: an extent of shaft rotation enabled by the stopper plate, a size of the joint components, a stopping force applied onto the shaft by the brake, and/or other structural and/or operational parameters.

In some embodiments, rotation joints and flexion joints differ from each other by an orientation of the joint assembly. For example, as shown in FIG. 5B, a rotation joint and a flexion joint (e.g. a shoulder rotation joint 591 and a shoulder flexion joint 593) are oriented such that their central shafts are substantially perpendicular to each other.

A potential advantage to separate joints (e.g. where each joint includes a brake) for limiting different types of movements may include that locking of a joint can be performed for any position and/or orientation of the input arm segments.

In an exemplary input arm structure, input arm joints are characterized by the following operational parameters (see joints of FIG. 5B):
Shoulder rotation joint 591: unlimited rotation (e.g. 360 degree rotation);
Shoulder flexion joint 593: flexion angle of up to 180 degrees;
a moment applied by the electromagnetic brakes of the shoulder (such as for each of the rotation joint and flexion joint): for example between 80 N-cm to 200 N-cm.
Elbow rotation joint 595: unlimited rotation (e.g. 360 degree rotation);
Elbow flexion joint 597: a flexion angle of up to 215 degrees;
a moment applied by the electromagnetic brakes of the elbow (such as for each of the rotation joint and flexion joint): for example between 30 N-cm to 70 N-cm.

In some embodiments, a moment of force applied on the brake when locked (such as a moment applied on a break portion which holds a rigid segment of the input arm) is determined, for example, according to the self-weight of one or both input arm segments that are connected at the joint, and/or by an axial extent of a segment relative to the joint (segment length). In an example, a moment acting on a joint brake is a function of the segment weight multiplied by a distance from the joint to a reference point along the segment, e.g. a central point or a most distal point from the joint. Optionally, a distance of one or more points along the rigid segment relative to a center of the joint (e.g. an axial center of the brake) is taken into account when determining the moment of force that the brake needs to withstand.

It is noted that the joint assembly described is provided as an example and should not be limited to the specific structure, for example, the joint assembly may include other types of brakes or in general any element configured to rotate along with a joint component (such as the shaft) and another element configured to limit or stop rotation of that component, e.g. by friction.

In some embodiments, additionally or alternatively to a brake, the input arm joint includes one or more motors. In some embodiments, each joint includes at least one motor. In some embodiments, pause mode (i.e. locking of the joint(s)) is entered by a control signal that stops motor actuation (such as via the local processors (such as PCBs) configured at the joint. In some embodiments, the control signal which stops the motors is initiated by pressing the pause button on the input arm handle.

### Exemplary Circuitry

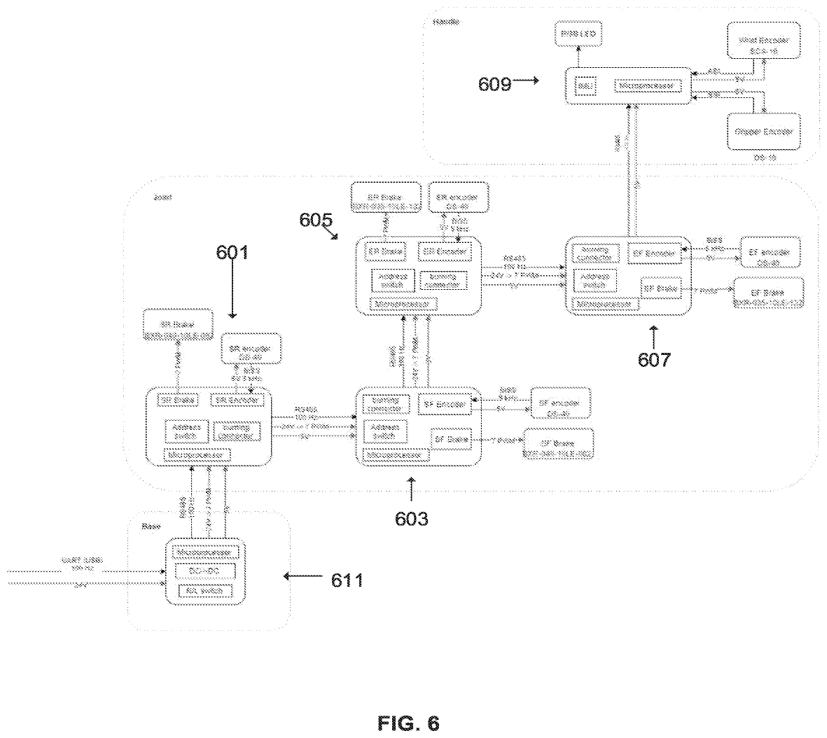

FIG. 6 is a block diagram of input arm circuitry, according to some embodiments.

In some embodiments, input arm control circuitry is divided into a plurality of control modules. Optionally, each control module is for a different input arm function and/or a different physical portion of the input arm. In some embodiments, each joint is controlled by a separate module.

In an example, a control module includes components such as a microprocessor, a switch for the transfer of power (such as power transfer to a brake), an encoder or a connector to an encoder.

In some embodiments, as shown in the example of this diagram, separate modules are provided as follows:
A module 601 for control of shoulder rotation (“SR”);
A module 603 for control of shoulder flexion (“SF”);
A module 605 for control of elbow rotation (“ER”);
A module 607 for control of elbow flexion (“EF”);
A module 609 for control of the proximal handle segment of the input arm (for example for control interfaces configured on that segment);
A base module 611 for control of power supply to the input arm, selection of input arm (e.g. between right and left arms), and/or other.

In some embodiments, input arm circuitry further includes one or more components for measuring and/or calculating acceleration, such as an accelerometer, inertial motion unit, gyro and/or others, as further described below.

### Alternative Input Arm Structure

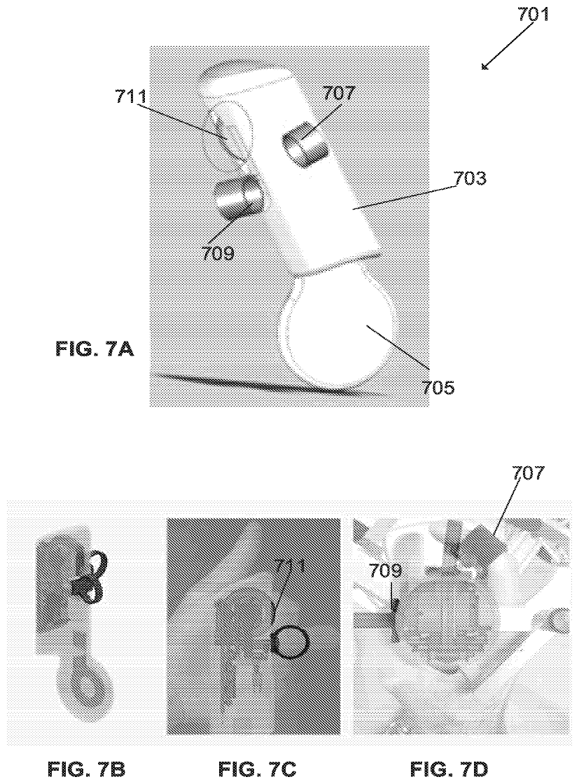

FIGS. 7A-D illustrate another example of a handle segment of an input arm for controlling movement of a surgical arm, according to some embodiments.

In some embodiments, an input arm handle 701 is comprised of a single rigid segment 703, which includes at a distal end thereof an attachment 705 to additional input arm segment(s).

In some embodiments, input arm handle 701 includes one or more interfaces, for example:
levers 707 and 709 which include engagement loops into a which a user gripping segment 703 inserts their fingers, for example, the thumb and index fingers (see for example FIG. 7D). In some embodiments, by pulling the loops away from the segment and approximating the loops back towards the segment control of an end-effector of the surgical arm is provided, for example, opening and closing of a gripper.
In some embodiments, movement of one or both levers (e.g. squeezing movement towards the segment) is configured to release locking of the arm, and/or enter resume mode (e.g. from a pause mode).
A roller button 711, for example mounted onto a body of segment 703, for control of one or more of: wrist joint rotation, pausing and/or locking the input arm, actuating linear movement of the surgical arm. In an example, different manipulations of the roller provide different controls: in some embodiments, pressing of the roller towards the segment locks the input arm and/or enters pause mode; in some embodiments, rolling of the roller rotates the wrist joint of the surgical arm; a tilted press on the roller generates linear movement of the surgical arm (e.g. advancing and retracting of the surgical arm). The above examples are listed in the table below:

| Action to be performed | Control | Action of user |
|---|---|---|
| Unlock JS, resume Arms | Master lever | Squeeze |
| | | And grips are matched |
| Close/open jaws | Master lever | Close |
| Lock JS, pause Arms | Roller | Straight Press |
| Wrist rotation | Roller | Rolling the roller's knob |
| Linear movement | Roller | Tilted press |

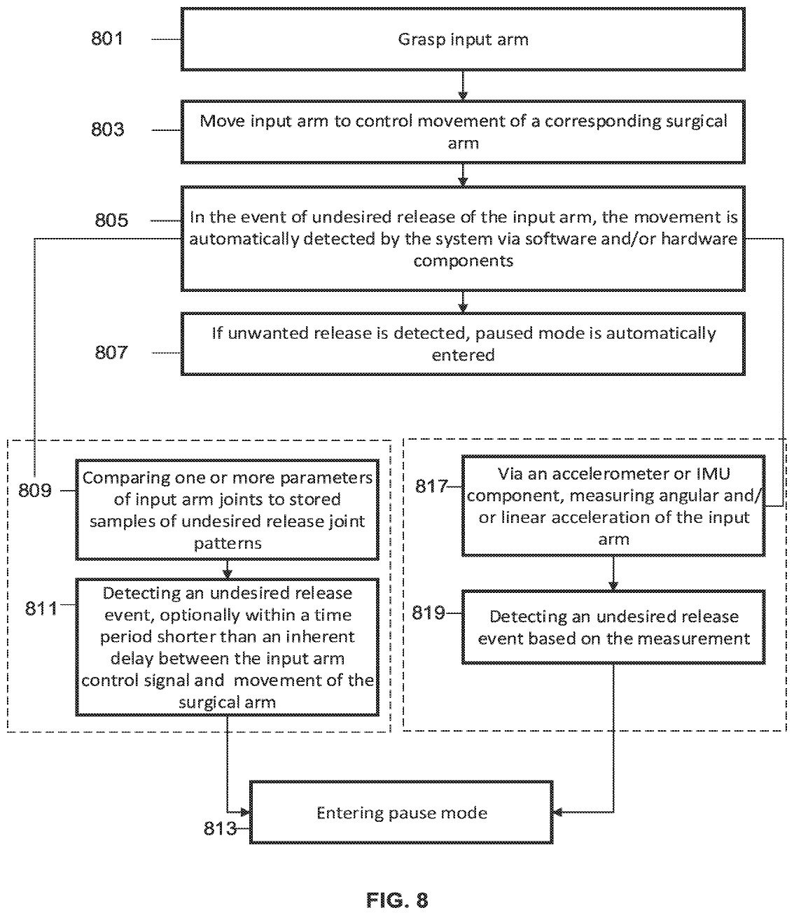

Input arm drop detection FIG. 8 is flowchart of a method for automatic detection of undesired release (e.g. drop) of the input arm by a user, according to some embodiments.

In some embodiments, it is desired to detect situations in which an input arm is accidently released by the user (for example, dropped, let go of without selectively entering pause mode, unintentionally released or pushed away from the user). In some embodiments, upon detection of undesired release of the input arm, movement of the corresponding surgical arm (which is controlled by the input arm) is immediately ceased. The following methods describe examples of automated detection of undesired release of the input arm.

Detection of undesired release may be potentially advantageous in use of an input arm as described, for example because the input arm is weighted and/or because the input arm comprises multiple segments (for example as compared to a single segment arm) implying that more portions of the input arm are induced to move (e.g. fall) when the user releases grip of the input arm.

In some embodiments, a user grasps the input arm (801) and moves the input arm to thereby move the corresponding surgical arm (803), where the surgical arm is optionally at least partially inserted into the body of the patient.

In some embodiments, in the event of undesired release of the input arm by the user, due to the effects of gravitation and as a result of the degrees of freedom of movement of the input arm joints and the weight of the input arm segments and joints, at least a portion of the input arm may fall (e.g. relative to its attachment to the base), optionally towards the ground and/or towards the user, and/or rotate in an uncontrolled manner. In some embodiments, a final position of the input arm following a drop is determined by the extent of movement enabled by each of the input arm joints. To cease such fall as closely as possible to when the fall started and/or to minimize an effect of a fall on surgical arm movement, in some embodiments, in the event of undesired release of the input arm, resulting motion (e.g. fall) of the input arm is automatically detected by the system (e.g. via software and/or hardware components (805), for example as further detailed below.

If unwanted release is detected, the system (e.g. a controller of the system) automatically activates pause mode of the input arm (807), optionally locking one or more of the input arm joints in place. Optionally, all joints are locked. Alternatively, a joint which was identified as the “source” of the fall (for example, measured movement of that joint indicated fall at a higher extent than movement of one or more other joints) is locked.

In some embodiments, movement of the corresponding surgical arm is ceased, thereby reducing or preventing damage (e.g. damage to tissue) which may have been caused if the surgical arm was to be moved according to the falling input arm. In some embodiments, if unwanted release is detected, the system notifies the user and optionally enters an “error mode”, with or without locking of the joints.

In some embodiments, according to one method of identifying undesired release, a current position of each input arm joint (e.g. as indicated by the one or more encoders of the joint, optionally relative to a calibrated “zeroed” position of the encoder) and/or a velocity of the joint and/or an acceleration of the joint are compared to known or simulated values, for example to a vector of samples (e.g. position samples, velocity samples, acceleration samples and/or other parameters) indicative of undesired release (809). In some embodiments, the samples include 3D coordinates of input arm joints which were recorded and/or measured and/or simulated during drop. In some embodiments, the samples were measured and/or simulated for each of the input arm joints separately. In an example, the samples include position of four input arm joints: a shoulder flexion joint, a shoulder rotation joint, an elbow flexion joint, an elbow rotation joint; optionally, the samples pertain to a position of all of the joints at a specific time point.

In some embodiments, current joint positions are sampled at a rate that is set according to a working frequency of the system, optionally being equal.

In some embodiments, comparing of the actual input arm joint positions (and/or velocity and/or acceleration) to the known (or predefined) samples is carried out using pattern recognition algorithms. In some embodiments, comparing is carried out using dynamic time warping algorithms, for example for identifying similarity between temporal sequences. A potential advantage of comparing using time warping algorithms may include that a match between coordinates can be detected even if there is a time gap of the position vectors being compared.

In some embodiments, comparing involves a cumulative analysis which checks a parameter (e.g. a position, velocity, acceleration) of each of at least two input arm joints and optionally of all input arm joints, and integrates the results to determine if undesired release has occurred.

In some embodiments, a cost function is implemented to determine similarity between actual joint parameters (e.g. position, velocity (optionally, absolute velocity, acceleration, and/or other movement related parameter) and the same parameter in measured or simulated samples. In some embodiments, a result of the cost function is compared with predefined threshold to determine whether similarity exists (indicating a fall).

In some embodiments, comparing involves determining a score for assessing if there is a match between current joint parameters (optionally, as measured over time) and the stored samples s. If the score is higher than a predetermined threshold, a correlation between the current joints parameters and the stored sample parameters may exist, indicating undesired release. In some embodiments, a score is calculated for each joints independently, and the cost function integrates the scores of multiple joints (optionally, of all input arm joints) to determine an undesired release event.

In some embodiments, detecting of undesired release is performed in real time. Optionally, a determination of undesired release is reached within a time period shorter than in inherent delay between the control signal generated by the input arm, and movement of the surgical arm itself (811). In an example, the determination is reached during and/or immediately following the fall, where the fall duration is about, about 50 msec, 70 msec, 90 msec or intermediate, shorter or longer time periods, while the delay between the control signal generated by the input arm and actual surgical arm movement is, for example, at least 100 msec, at least 150 msec, at least 250 msec or intermediate, longer or shorter delay. Therefore, in most situations of undesired release, fall of the input arm may not affect the surgical arm or only slightly move the surgical arm, for example, will not cause large scale surgical arm movements.

In some embodiments, due to that the comparison requires a sufficient amount of data of joint positions to be collected, the process of detecting undesired release may start only a certain amount of time after the actual fall had begun. In an example, the input arm starts to fall, a determination is reached that the input arm is falling, and then the input arm is stopped (e.g. by automatically entering pause mode), optionally before the input arm reaches a final position that it would have reached if it was not stopped.

In some embodiments, once a determination of undesired release is reached, pause mode is automatically entered (813). Optionally, all input arms joints simultaneously lock. In some embodiments, the system automatically stops the input arm signals from generating movement of the surgical arm.

In some embodiments, the system is configured to return the input arm to a last desired position, for example being a position detected before the identifying of fall of the arm. Additionally or alternatively, following an undesired release event, the user moves to the input arm to a calibration position in order to resume and/or directly resumes via the dedicated interface (e.g. the button on the input arm proximal segment). In some embodiments, following an event of undesired release, the input arm needs to be aligned to match a current surgical arm position in order to resume controlled movement.

In some embodiments, according to a second method of identifying undesired release, optionally performed independently or together with the first method, an accelerometer and/or other inertial motion unit component measure acceleration of the input arm or portions of the arm. For example, angular and/or linear acceleration of the input arm is measured (817). If the measured value is above a predefined threshold, a determination of undesired release is made (819).

In some embodiments, a cumulative result of the IMU based method and the software based method is obtained. Optionally, a cost function is implemented to calculate a total score for a given input arm position vector (which includes, for example, a plurality of joint positions measured over time), where the total score is calculated based on the IMU measurement and the pattern detection comparison, to determine whether an undesired release event occurred.

Once the determination is made, pause mode is automatically entered (813).

In some embodiments, an IMU component is used for restricting a range of input arm movements and/or a speed of movement. For example, if acceleration passes a threshold, a user may be alerted that a speed of movement is too high or that the movement they are trying to achieve is not possible. Additionally or alternatively, if acceleration passes a threshold, the system automatically slows and/or stops and/or limits the range of movement, for example by locking at least one of the input arm joints.

In some embodiments, following detection of an undesired release event the system (e.g. system controller) automatically recalibrates the input arm(s) and/or the surgical arm(s), optionally aligning them with respect to each other.

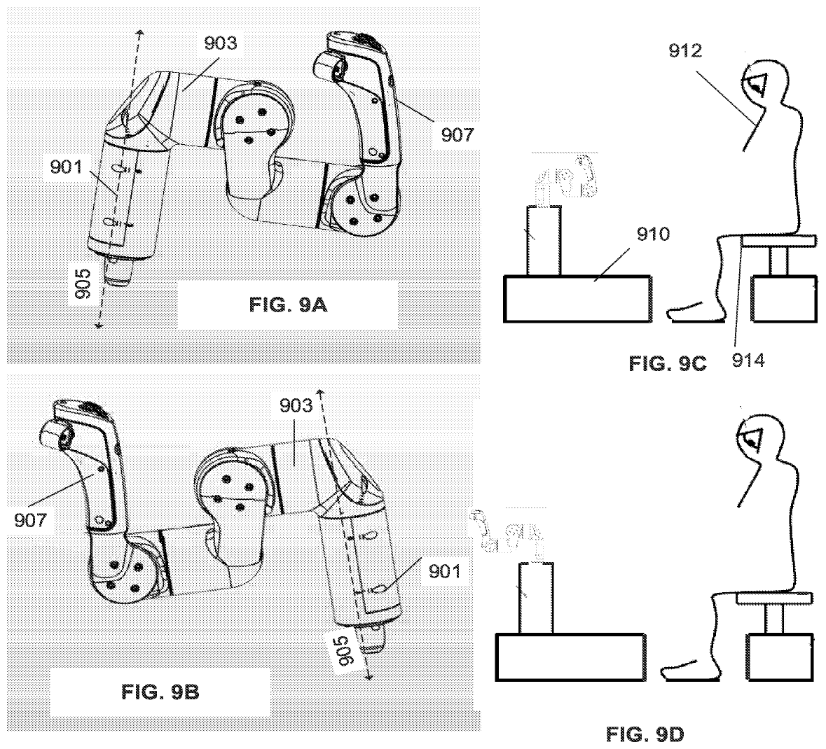

FIGS. 9A-D are examples of input arm configurations, according to some embodiments. In some embodiments, the input arm is configured to extend from its support segment 901 in two or more different directions, for example, with at least a first rigid segment 903 of the input arm extending at different angles with respect to a long axis 905 of the support segment.

FIGS. 9C-D show the exemplary input arm configurations as installed on a control console 910, where a user 912 using a user support, e.g. sitting on a seat 914, manipulates the input arm(s).

In the example of FIGS. 9A, 9C, segment 903 extends at a 90 degree angle relative to the long axis 905 of the support segment; in the example of FIGS. 9B, 9D, segment extends at a 270 degree angle relative to long axis 905 of the support segment.

In some embodiments, a handle segment 907 is configured to face the user in all input arm orientations relative to the input arm support. Optionally, the handle segment is in an upright orientation (e.g. facing a direction opposite the floor) to facilitate grasping by a user. In some embodiments, due to the handle segment extending towards the user (for example towards a user support of the control console), the user can easily access and grasp the handle at all input arm orientations. Optionally, the handle segment remains directed towards a proximal end of the control console (e.g. in an elongate control console) at all input arm orientations relative to the input arm support.

In some embodiments, the input arm is attached by a pivotable joint to its support segment, allowing the arm to be rotated relative to the support long axis. In some embodiments, the support segment (along with the rest of the input arm) is detachable from the control console (e.g. from a control console platform) and can be manually placed (for example, during preparation for surgery) in the selected orientation. In an example, the base comprises a recess (not shown) shaped to receive an end of the support segment. Optionally, the recess includes one or more inner protrusions for aligning the support segment such that the input arm extends in a desired orientation (e.g. selected from 2, 3, 4, 5, 6, or intermediate, larger or smaller number of possible orientations).

Different input arm configurations where the input arm (or portions of it) extend at different angles relative to the support may be advantageous in that a configuration can be selected based on the surgical approach, to match the manner in which the surgical arm is oriented and used. Optionally, the configuration is selected in accordance with a view of the surgical arm by a camera, to match the user's view of the input arm. In an example, the configuration of FIG. 9A is advantageous for transvaginal procedures, for example when the surgical arms are inserted through the vagina and are then retroflected into the intraperitoneal space, while the camera is inserted, for example, through the naval or abdominally and towards vagina; optionally, once the surgical arms are retroflected, the camera field of view is similar to a direction of extension of the surgical arms; and the configuration of FIG. 9B is advantageous for abdominal procedures, where the surgical arms are inserted through an abdominal port and the camera is inserted, for example, through the naval or abdominally.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of operation of an input arm for control of a surgical mechanical arm, the input arm comprising a plurality of rigid segments coupled to each other by joints, at least one of said plurality of rigid segments shaped and sized to be at least partially held by a user's hand, the method comprising:
grasping the input arm and manipulating the input arm by moving the rigid segments relative to each other; wherein during said grasping and manipulating, electrical power is supplied to at least one brake configured in each of said joints to unlock said brake from a normally locked state; and
at any selected position of said plurality of rigid segments, entering a pause mode by ceasing said electrical power supply to said at least one brake in each of said joints to thereby lock each of said joints and maintain said plurality of rigid segments in place.

2. The method according to claim 1, further comprising resuming movement of said input arm directly from said selected position in which said input arm was locked.

3. The method of claim 2, wherein said resuming comprises re-establishing electrical power supply to said brake of each of said joints to release said brake.

4. The method according to claim 1, wherein sequential rigid segments are coupled to each other by one or both of: a flexion joint which is configured to provide for flexion of said coupled rigid segments relative to each other, and a rotation joint which is configured for rotation of each rigid segment about a rigid segment long axis, and wherein each of said flexion joint and rotation joint is independently controllable and lockable.

5. The method according to claim 2, wherein said entering a pause mode comprises actuating a first interface of said input arm, and wherein said resuming comprises actuating a second interface of said input arm, wherein said first interface and said second interface include different interface types.

6. The method according to claim 5, wherein said first interface comprises a push button and said second interface comprises a squeezable lever.

7. An input arm for control of a surgical mechanical arm, the input arm comprising:
a plurality of rigid segments, at least one of said plurality of rigid segments shaped and sized to be at least partially held by a user's hand;
a plurality of joints, which each joint couples between sequential rigid segments;
wherein each of said joints comprises a brake which changes configuration between a locking configuration in which relative movement of said sequential rigid segments is prevented and an unlocked configuration in which relative movement of said sequential segments is allowed; wherein said change in configurations is controlled via electrical power supply to said electromechanical brake; and
a controller configured to control said electrical power supply to said electromechanical brake to lock or unlock said joint.

8. The input arm according to claim 7, wherein said plurality of joints comprise at least two joints and wherein said controller is configured to control electrical power supply to said at least two joints simultaneously.

9. The input arm according to claim 7, wherein said brake is normally locked and wherein said controller is configured to enable electrical power supply to unlock said brake.

10. (canceled)

11. The input arm according to claim 8, wherein said input arm comprises at least 5 rigid segments connected to each other by at least 4 joints.

12. The input arm according to claim 7, wherein said brake comprises at least two portions that are magnetically attracted, and wherein when said electrical power supply is enabled, one of said portions is held away from the other portion, allowing for movement of the other portion.

13. An input arm according to claim 7, wherein said surgical mechanical arm includes an end-effector having portions that are movable with respect to each other, and wherein said input arm comprises
a lever mounted onto said at least one of said rigid segments which is held by the user's hand, being configured to slide along a designated track configured on a surface of said rigid segment, to actuate distancing and approximating of said end-effector portions to each other,

14-17. (canceled)

18. An input arm according to claim 7, wherein the brake comprises at least two portions, where a first portion is coupled to said shaft and rotates along with said shaft and a second portion is positioned and configured to stop rotation of said first portion along with shaft when said second portion is approximated towards said first portion such that friction contact is formed.

19-22. (canceled)

23. A method of determining undesired release of an input arm which controls movement of a surgical mechanical arm, the input arm comprising a plurality of rigid segments coupled to each other by joints, the method comprising:
assessing one or more parameters of at least one joint of the input;
comparing the assessed joint parameters to a set of stored samples, the stored samples being indicative of undesired release patterns of the joint; wherein said comparing comprises scoring said assessed joint parameters based on their correlation to said stored samples;
based on said comparing, determining undesired release and entering a pause mode in which said at least one joint locks to prevent relative movement of said rigid segments.

24. The method according to claim 23, wherein said one or more parameters include:
a position of the joint, a velocity of movement of the joint, acceleration of the joint.

25. The method according to claim 23, comprising assessing and comparing said one or more parameters for all input arm joints.

26. The method according to claim 23, wherein said assessing comprises receiving position data from at least one encoder of said at least one joint.

27. The method according to claim 23, wherein said determining is finalized within a time period shorter than a time delay between control signals generated by said input arm and actual movement of said surgical mechanical arm.

28. The method according to claim 23, wherein said comparing comprises implementing a time warping algorithm to which said assessed one or more parameters are inputted.

29. The method according to claim 23, wherein said input arm comprises at least one inertial measurement unit embedded within said input arm or mounted on it, and wherein said scoring takes into account acceleration measured by said inertial measurement unit.

30-31. (canceled)

32. The input arm of claim 14, wherein a degree of distancing of said end effector portions is set by an axial extent of said lever relative to said track, and wherein said degree of distancing remains constant as long as said lever is not moved on said track.

33. An input arm according to claim 18, wherein said input arm comprises a disc shaped encoder configured to detect a rotational orientation of said shaft, wherein said brake and said disc shaped encoder are co-axially stacked on said central elongate shaft, such that said central elongate shaft extends through a central opening formed in said brake and in said disc shaped encoder.
